SEQUENCE LISTING (12 sequences)

SEQ ID NO: 1 - DNA, 23 bp, artificial sequence
  CLDN1 probe
caggctctcttcactggctgggc

SEQ ID NO: 2 - DNA, 20 bp, artificial sequence
  CLDN1 forward primer
ccagtcaatgccaggtacga

SEQ ID NO: 3 - DNA, 20 bp, artificial sequence
  CLDN1 reverse primer
gaaggcagagagaagcagca

SEQ ID NO: 4 - DNA, 25 bp, artificial sequence
  RPL32 probe
aattaagcgtaactggcggaaaccc

SEQ ID NO: 5 - DNA, 21 bp, artificial sequence
  RPL32 forward primer
gcaccagtcagaccgatatgt

SEQ ID NO: 6 - DNA, 20 bp, artificial sequence
  RPL32 reverse primer
accctgttgtcaatgcctct

SEQ ID NO: 7 - DNA, 26 bp, artificial sequence
  E gene Sarbeco probe
acactagccatccttactgcgcttcg

SEQ ID NO: 8 - DNA, 26 bp, artificial sequence
  E gene Sarbeco forward primer
acaggtacgttaatagttaatagcgt

SEQ ID NO: 9 - DNA, 22 bp, artificial sequence
  E gene Sarbeco reverse primer
atattgcagcagtacgcacaca

SEQ ID NO: 10 - DNA, 25 bp, artificial sequence
  RdRP SARS CoV2 probe
caggtggaacctcatcaggagatgc

SEQ ID NO: 11 - DNA, 22 bp, artificial sequence
  RdRP SARS CoV2 forward primer
  misc_feature (5)..(5): R = G or A
gtgaratggtcatgtgtggcgg

SEQ ID NO: 12 - DNA, 26 bp, artificial sequence
  RdRP SARS CoV2 reverse primer
  misc_feature (3)..(3): R = G or A
  misc_feature (12)..(12): S = G or C
caratgttaaasacactattagcata

(19) United States
(12) Patent Application Publication
(10) Pub. No.: US 2023/0151442 A1
(43) Pub. Date: May 18, 2023

(54) RAPID DETECTION OF VIRAL INFECTION USING RT-PCR

(71) Applicant: Bayer Aktiengesellschaft, Leverkusen (DE)

(72) Inventors: Stefan GOLZ, Mülheim an der Ruhr (DE); Holger SUMMER, Wuppertal (DE); Silke AIGNER, Oberhausen (DE); Heidrun ELLINGER-ZIEGELBAUER, Haan (DE); Kirsten LEINEWEBER, Velbert-Langenberg (DE); Thomas MÜLLER, Langenfeld (DE)

(73) Assignee: Bayer Aktiengesellschaft, Leverkusen (DE)

(21) Appl. No.: 17/917,512

(22) PCT Filed: Apr. 1, 2021
(86) PCT No.: PCT/EP2021/058720

§ 371 Date: Oct. 6, 2022

Related U.S. Application Data
(60) Provisional application No. 63/007,255, filed on Apr. 8, 2020.

(51) Int. Cl. C12Q 1/70; C12Q 1/6848

(52) U.S. Cl. CPC C12Q 1/701; C12Q 1/6848

(57) ABSTRACT

A lysis buffer comprising one non-ionic surfactant is provided which can be used as a one-step reagent of the preparation, storage, amplification, and/or detection of nucleic acids. Various embodiments of the lysis buffer of the invention comprise other substances that are compatible or useful in lysing cells, storing nucleic acids, amplifying nucleic acids, purifying nucleic acids, detecting nucleic acids, and/or other procedures for analysis of nucleic acids. Methods and kits based on the lysis buffer are also provided, including those for rapid lysis of cells and direct use of the resulting cell lysates in RT-PCR.

### REFERENCE TO SEQUENCE LISTING SUBMITTED ELECTRONICALLY

The sequence listing provided with the application via electronic filing is part of the specification and is herein incorporated by reference in its entirety.

### BACKGROUND

### 1. Field

The present disclosure relates to an improved method for the detection of viral RNA from a biological sample using reverse transcription-polymerase chain reaction (RT-PCR).

Some representative RT-PCR methods for detection of viral RNA require lysis of a biological sample in lysis buffer containing harsh chemicals under highly denaturing conditions to inactivate RNases and stabilize RNA followed by isolation of the RNA using column purification to remove these chemicals from the lysis buffer, which can interfere with the subsequent RT-PCR. For example, the lysis buffers included in the standard TRIzol RNA preparation kits typically contains components such as guanidinium thiocyanate, phenol, and/or chloroform, which degrades or denatures proteins such as polymerases and would therefore interfere with the subsequent PCR reactions.

Some disadvantages of these methods are the multiple steps, lack of sensitivity, increased time, increased costs, and reagents needed to obtain results.

A solution to this technical problem is provided by the embodiments characterized in the claims.

### BRIEF SUMMARY

The present application provides compositions that are suitable for lysis of cells and/or viruses and analysis of nucleic acids.

In some embodiments, the present application provides a lysis buffer comprising one non-ionic surfactant. Various embodiments of the lysis buffer of the invention comprise other substances that are compatible or useful in lysing cells and/or viruses, storing nucleic acids, amplifying nucleic acids, purifying nucleic acids, detecting nucleic acids, and/or other procedures for analysis of nucleic acids. In some embodiments, the lysis buffer can be considered a one-step reagent of the preparation, storage, amplification, and/or detection of nucleic acids.

Methods and kits based on the compositions are also provided, including those for rapid lysis of cells and direct use of the resulting cell lysates in RT-PCR.

In some embodiments, the present application provides a method of lysing at least one cell and/or at least one virus using the lysis buffer of the invention. The method of lysing generally comprises contacting at least one cell or at least one virus with the lysis buffer of the invention for a sufficient amount of time to cause the cell or virus to lyse. In various embodiments, additional optional steps are included in the lysis method, such as storing the lysate for a period of time prior to use of the lysate. Likewise, other exemplary additional steps can include amplifying one or more nucleic acids in the cell lysate, and/or detecting the nucleic acid(s).

It has surprisingly been found that a single buffer of the invention can be suitable for both lysis of cells and viruses and can be present as a sub-component of reactions leading to amplification and detection of nucleic acids. For instance, the buffer of the invention can be suitable to lyse a cell, such as a human or animal cell, infected with a virus. The obtained lysate may be directly used as template in subsequent reactions leading to amplification, detection and quantification of nucleic acids. In another embodiment, the present application provides a method of amplifying one or more nucleic acids. In an aspect, the method comprises contacting a biological sample with the lysis buffer of the invention to produce a lysate, and amplifying a nucleic acid in the lysate. In various embodiments, amplifying a nucleic acid is by a PCR method, such as qPCR, RT-PCR, or RT-qPCR. In certain embodiments, one or more control reactions are included, such as a control reaction to permit normalization of the amount of nucleic acid being amplified with respect to other amplification reactions that are being performed concurrently, or with respect to a standard amplification curve.

The present application also provides kits. In general, the kits contain the lysis buffer of the invention. The kits can further comprise one or more substances, materials, reagents, etc. that can be used for lysis of cells or viruses, storage of nucleic acids or lysates, be present during amplification of nucleic acids, or detection or quantification of nucleic acids. In embodiments, some or all of the materials, reagents, etc. necessary to lyse cells or viruses, amplify nucleic acids, and/or detect and/or quantify nucleic acids are included in the kit.

Use of the lysis buffer and kits in a method for amplifying one or more nucleic acids comprising contacting a sample with the lysis buffer to produce a lysate and amplifying at least one nucleic acid in the lysate is also provided.

Use of the lysis buffer and kits in a method to detect an RNA or DNA virus comprising contacting a sample with the lysis buffer to produce a lysate, and in case of RNA virus detection reverse transcribing RNA within the lysate to obtain cDNA, and amplifying at least one nucleic acid from an RNA virus in the lysate using a set of primers derived from the RNA virus is also provided.

In one aspect the present invention relates to a method to detect an RNA or DNA virus comprising contacting at least one sample with a lysis buffer to produce a lysate, in case of RNA virus detection reverse transcribing RNA within the lysate to obtain cDNA, and amplifying at least one nucleic acid from an RNA or DNA virus in the lysate using a set of primers derived from a nucleic acid sequence of the RNA or DNA virus.

In particular embodiments, the lysis buffer comprises at least one non-ionic surfactant, glycerol, and at least one salt.

In particular embodiments, the at least one non-ionic surfactant has a hydrophilic polyethylene oxide chain and an aromatic hydrocarbon lipophilic or hydrophobic group.

In particular embodiments, the at least one non-ionic surfactant is Triton X-100.

In particular embodiments, the lysis buffer comprises a non-ionic surfactant in an amount from about 0.05% to about 20%.

In particular embodiments, the salt is disodium phosphate.

In particular embodiments, the lysis buffer further comprises one or more of the following: Tris-HCl, dithiothreitol (DTT), RNase-free water, RNase inhibitor, and mixtures thereof.

In particular embodiments, the pH of the lysis buffer is from about 7.5 to about 8.5.

In particular embodiments, the RNA virus is a coronavirus.

In particular embodiments, the coronavirus is SARS CoV-2.

In particular embodiments, the coronavirus is a variant of SARS CoV-2.

In particular embodiments, the set of primers is directed to a viral RNA gene selected from the group consisting of an E gene, an N gene, and an RdRP gene.

In particular embodiments, the set of primers is directed to the E gene and comprise or consist of SEQ ID NO: 8 and/or SEQ ID NO: 9, or a complement thereof.

In particular embodiments, the set of primers is directed to the RdRP gene and comprise or consist of SEQ ID NO: 11 and/or SEQ ID NO: 12, or a complement thereof.

In particular embodiments, the method does not comprise an RNA extraction step.

In particular embodiments, the lysate is directly used in the reverse transcription.

In particular embodiments, the sample is a biological sample.

In particular embodiments, the biological sample is collected using a swab.

In particular embodiments, the biological sample is a nasal swab.

In particular embodiments, the at least one sample comprises at least one human cell.

In particular embodiments, the sample is placed in 100 μl of the lysis buffer.

In particular embodiments, the method further comprises incubating the mixture of the lysis buffer and the sample for 5 minutes at room temperature to produce a lysate.

In particular embodiments, the method further comprises centrifuging the lysate and collecting the supernatant.

In particular embodiments, the lysate is centrifuged at 12,000 rpm for 2 minutes at room temperature.

In a further aspect, the present invention relates to a method for amplifying one or more nucleic acids comprising contacting at least one sample with a lysis buffer to produce a lysate, and amplifying at least one nucleic acid in the lysate, wherein the lysis buffer comprises at least one non-ionic surfactant, glycerol, and at least one salt.

In particular embodiments, the at least one non-ionic surfactant has a hydrophilic polyethylene oxide chain and an aromatic hydrocarbon lipophilic or hydrophobic group.

In particular embodiments, the at least one non-ionic surfactant is Triton X-100.

In particular embodiments, ein the lysis buffer comprises a non-ionic surfactant in an amount from about 0.05% to about 20%.

In particular embodiments, the salt is disodium phosphate.

In particular embodiments, the lysis buffer further comprises one or more of the following: Tris-HCl, dithiothreitol (DTT), RNase-free water, RNase inhibitor, and mixtures thereof.

In particular embodiments, the at least one sample is a biological sample.

In particular embodiments, the biological sample is collected using a swab.

In particular embodiments, the biological sample is a nasal swab.

In particular embodiments, the sample comprises at least one human cell.

In particular embodiments, the sample is placed in 100 μl of the lysis buffer.

In particular embodiments, the method further comprises incubating the mixture of the lysis buffer and the sample for 5 minutes at room temperature to produce a lysate.

In particular embodiments, the method further comprises centrifuging the lysate and collecting the supernatant.

In particular embodiments, the lysate is centrifuged at 12,000 rpm for 2 minutes at room temperature.

In particular embodiments, the amplifying at least one nucleic acid in the lysate is performed by PCR, qPCR, RT-PCR, or RT-qPCR.

In particular embodiments, the amplifying at least one nucleic acid in the lysate is performed by one-step RT-qPCR.

In particular embodiments, the method does not comprise an RNA extraction step.

In particular embodiments, the lysate is directly used in the amplifying.

In particular embodiments, the at least one nucleic acid is from an RNA virus.

In a further aspect, the present invention relates to a method for the identification of a subject infected with SARS CoV-2 or a variant of SARS CoV-2 comprising obtaining a lysate from a biological sample obtained from the subject, reverse transcribing RNA within the lysate to obtain cDNA, and subjecting the cDNA to PCR assay using a set of primers derived from a nucleotide sequence of the SARS CoV-2 genome or the genome of the variant of SARS CoV-2.

In particular embodiments, the lysate is obtained by contacting the biological sample with a lysis buffer comprising at least one non-ionic surfactant, glycerol, and at least one salt.

In particular embodiments, the method does not comprise an RNA extraction step.

In particular embodiments, the lysate is directly used in the reverse transcription.

In a further aspect, the present invention relates to a method of amplifying, identifying, detecting, and/or analyzing a target nucleic acid, comprising contacting a sample with a lysis buffer to produce a lysate, reverse transcribing RNA within the lysate to obtain cDNA, and subjecting the cDNA to a PCR assay using a set of primers directed to the target nucleic acid.

In particular embodiments, the lysate is obtained by contacting the sample with a lysis buffer comprising at least one non-ionic surfactant, glycerol, and at least one salt.

In particular embodiments, the method does not comprise an RNA extraction step.

In particular embodiments, the lysate is directly used in the reverse transcription.

In a further aspect, the present invention relates to a kit comprising a lysis buffer, wherein the lysis buffer comprises at least one non-ionic surfactant, glycerol, and at least one salt.

In particular embodiments, the kit further comprises at least one reagent for amplification of a target nucleic acid.

In particular embodiments, the kit comprises at least one primer and/or at least one probe for amplification of a target nucleic acid.

In particular embodiments, the kit is a kit for detection of RNA using one-step RT-qPCR.

In particular embodiments, the kit comprises one or more of at least one reverse transcriptase, at least one DNA polymerase, an RNase inhibitor, nucleotides, primers, probes, labels, or any combination thereof.

In particular embodiments, the target nucleic acid is derived from a RNA virus.

In particular embodiments, the RNA virus is a coronavirus.

In particular embodiments, the RNA virus is SARS CoV-2.

In particular embodiments, the kit comprises a set of primers selected from SEQ ID NO: 8 and/or SEQ ID NO: 9, or a complement thereof and SEQ ID NO: 11 and/or SEQ ID NO: 12, or a complement thereof.

In a further aspect, the present invention relates to a lysis buffer comprising 1-5% Triton X-100 and one or more of the following components: 5-20% glycerol, 0.5-4 mM DTT, 10-50 mM Na2HPO4, and 10-50 mM Tris-HCl.

In particular embodiments, the lysis buffer comprises about 3% Triton X-100 and one or more of the following components: about 10% glycerol, about 2 mM DTT, about 25 mM Na2HPO4, and about 25 mM Tris-HCl.

In particular embodiments, the pH of the lysis buffer is from about 7.5 to about 8.0.

In particular embodiments, the lysis buffer further comprises RNase-free water.

In particular embodiments, the RNase-free water is included in an amount from about 1:15 to 1:1 (lysis buffer:RNase-free water).

In particular embodiments, the lysis buffer further comprises an RNAse inhibitor.

In a further aspect, the present invention relates to the use of the lysis buffer according to the present invention in a method for amplifying one or more nucleic acids comprising contacting at least one sample with the lysis buffer to produce a lysate, and amplifying at least one nucleic acid in the lysate.

In a further aspect, the present invention relates to the use of the lysis buffer according to the present invention in a method to detect an RNA virus comprising contacting at least one sample with the lysis buffer to produce a lysate, reverse transcribing RNA within the lysate to obtain cDNA, and amplifying at least one nucleic acid from an RNA virus in the lysate using a set of primers derived from a nucleic acid sequence of the RNA virus.

### BRIEF DESCRIPTION OF THE DRAWINGS

The patent or application file contains at least one drawing executed in colour. Copies of this patent or patent application publication with colour drawing(s) will be provided by the Office upon request and payment of the necessary fee.

For a further understanding of the nature, objects, and advantages of the present disclosure, reference should be had to the following detailed description, read in conjunction with the following drawings, wherein like reference numerals denote like elements.

FIG. 1 shows amplification curves from RT qPCR for the epithelial marker CLDN1 and the human housekeeping gene RPL32 in samples obtained from nasal swabs of ten human, healthy volunteers (S1-S10), swab without sample (swab only, neg. control), and sample without template (NTC, neg. control). Red: RPL32; blue: CLDN1. Reactions for each sample were performed in quadruplicate.

FIG. 2 provides a summary of the data illustrated in FIG. 1. For each sample (S1-S10 and negative controls), the left bar present CLDN1 detection and the right bar presents RPL32 detection. The cycle threshold (CT) or crossing point (CP) is the number of cycles during the PCR amplification until the measured fluorescence reaches a value where the fluorescence can be distinguished from the background fluorescence.

FIG. 3 shows amplification curves illustrating sensitivity of RT qPCR for detection of a standard RNA extraction viral positive control (EAV).

FIG. 4 shows amplification curves illustrating sensitivity of RT qPCR for detection of SARS CoV-2 E-gene.

FIG. 5 shows amplification curves illustrating sensitivity of RT qPCR for detection of SARS CoV-2 N-gene.

FIG. 6 shows amplification curves illustrating sensitivity of RT qPCR for detection of SARS CoV-2 RdRP gene.

FIG. 7 shows amplification curves demonstrating that SARS CoV-2 genes (E-gene and RdRP gene) can be detected in nasal swabs from ten healthy human volunteers (S1-S10) spiked with synthetic SARS CoV-2 marker oligonucleotides with a detection of <20 copies. Reactions for each sample were performed in quadruplicate.

FIG. 8 provides a summary of the data illustrated in FIG. 7. For each sample (S1-S10 and negative controls), the left bar present E-Gene detection and the right bar presents RdRP-Gene detection. NTC=no template control; PosControl RNA=controls without lysates. The cycle threshold (CT) or crossing point (CP) is the number of cycles during the PCR amplification until the measured fluorescence reaches a value where the fluorescence can be distinguished from the background fluorescence.

### DETAILED DESCRIPTION

In an aspect, the disclosure provides for methods of amplifying, identifying, detecting, quantifying and/or analyzing a target nucleic acid, comprising, for example:
(a) obtaining a sample, optionally comprising no cells or one or more cells, for example epithelial cells,
(b) contacting the sample with a lysis buffer of the invention to produce a lysate,
(c) optionally freezing the lysate,
(d) thawing the lysate, if necessary, and centrifuging the lysate, if necessary
(e) collecting and transferring the supernatant to a new reaction vessel, such as a reaction tube or microtiter plate, if centrifugation is necessary
(f) optionally freezing the supernatant,
(g) thawing the supernatant, if necessary, and
(h) amplifying, identifying, detecting and/or analysing a target nucleic acid by any suitable means.

In some embodiments, the amplifying, identifying, detecting, and/or analysing a target nucleic acid is carried out using a PCR technique, such as, for example, PCR, qPCR, RT-PCR, RT-qPCR. Methods for PCR are well known, and any suitable method can be used in the methods of the invention.

In an aspect, the amplifying, identifying, detecting, quantifying and/or analysing a target nucleic acid comprises, for example:
(1) adding a PCR MasterMix, for instance a qPCR MasterMix, to a lysate obtained by lysing at least one virus and/or one or more cells from a subject, for example epithelial cells, in a qPCR plate,
(2) sealing the qPCR plate with clear qPCR foil,
(3) centrifuging the qPCR plate,
(4) performing PCR, for instance qPCR, and
(5) analysing the data.

In an aspect, any suitable PCR MasterMix can be used. Several commercial PCR MasterMix formulations, including qPCR MasterMix formulations, are available and can be used in the instant invention. Alternatively, a PCR MasterMix can be created by mixing components necessary for a PCR reaction to occur, such as a DNA polymerase, nucleotides, and magnesium, and optionally a reverse transcriptase and RNase inhibitor. In some embodiments, the PCR MasterMix and the lysate are added in a 5:1 ratio (PCR MasterMix:lysate).

In an aspect, the qPCR is a one-step RT-PCR using a LightCycler® 480 QPCR reader (Roche). However, any suitable PCR machine may be used in the methods of the invention.

In an aspect, the data are analysed using LightCycler® 480 software. However, any suitable software may be used to analyse the PCR data.

In an aspect, the assays described herein are sensitive enough to detect about 20 copies or less, about 10 copies or less, about 5 copies or less, about 4 copies or less, about 3 copies or less, or about 2 copies or less of a target nucleic acid within a sample.

As used herein, the meaning of “surfactant” is the broadest definition that is readily recognized by a person of ordinary skill in the art. That is, surfactants are wetting agents that lower the surface tension of a liquid and/or lower the interfacial tension between two liquids. A surfactant that does not have a positive or negative charge in water, yet is soluble in water, is a “non-ionic surfactant”. Combinations of two or more non-ionic surfactants are encompassed within the term “non-ionic surfactant”.

The present invention features, in one aspect, a lysis buffer. It has surprisingly been shown that the lysis buffer of the invention is suitable for lysis of cells and viruses and can be included as a component of reaction mixtures for amplification of nucleic acids by PCR methods, such as reverse transcriptase PCR (RT-PCR) and quantitative PCR (qPCR).

The lysis buffer comprises at least one detergent capable of disrupting membranes such as cell membranes and virus membranes. In some embodiments, the lysis buffer comprises at least one non-ionic surfactant. In some embodiments, the at least one non-ionic surfactant has a hydrophilic polyethylene oxide chain and an aromatic hydrocarbon lipophilic or hydrophobic group. Suitable non-ionic surfactants include, but are not limited to, Triton X-100, Igepal CA-630 (Nonidet P-40), Conco NI, Dowfax 9N, Igepal CO, Makon, Neutronyx 600's, Nonipol NO, Plytergent B, Renex 600's, Solar NO, Sterox, Serfonic N, T-DET-N, Tergitol NP, Triton N, BIGCHAP (N,N-bis-(3-DGluconamidopropyl)cholamide) or deoxy-BIGCHAP (N,N-bis(3-Gluconamidopropyl) deoxycholamide); Decanoyl-N-methylglucamide; n-Decyl α-DGlucopyranoside; n-Decyl β-D-Glucopyranoside; n-Decyl β-D-Maltopyranoside; Digitonin; n-Dodecyl β-D-Glucopyranoside; n-Dodecyl α-D-Maltoside; n-Dodecyl β-D-Maltoside; heptanoyl-N-methylglucamide; n-Heptyl β-D-Glucopyranoside; N-Heptyl β-D-Thioglucopyranoside; n-Hexyl β-D-Glucopyranoside; 1-Monooleoyl-rac-glycerol; Nonanoyl-N-methylglucamide; n-Nonyl α-D-Glucopyranoside; n-Nonyl β-D-Glucopyranoside; Octanoyl-N-methylglucamide; n-Octyl α-D-glucopyranoside; n-Octyl β-D-Glucopyranoside; Octyl β-D-Thiogalactopyranoside; Octyl β-D-Thioglucopyranoside; Polyoxyethylene Esters (such as 8-stearate polyoxyethylene ester (Myrj 45), 40-stearate polyoxyethylene ester (Myrj 52), 50-stearate polyoxyethylene ester (Myrj 53), and 100-stearate polyoxyethylene ester (Myrj 59)), Polyoxyethylene Ethers (such as those containing one or more ethyl groups, methyl groups, pentyl groups, cetyl groups, stearyl groups, oleyl groups, hexyl groups, octyl groups, decyl groups, lauryl groups, myristyl groups, heptyl groups, tridecyl groups, isohexadecyl groups, and combinations thereof); Polyoxyethylenesorbitan esters (such as those containing one or more monolaurate groups, monooleate groups, monopalmitate groups, monostearate groups, trioleate groups, and tristearate groups, and combinations thereof, including, but not limited to the “Tween” series of detergents); Sorbitan esters (such as those containing one or more monolaurate groups, monooleate groups, monopalmitate groups, monostearate groups, sesquioleate groups, trioleate groups, tristearate groups, and combinations thereof); Tergitol; n-Tetradecyl β-D-Maltoside; the Triton series of detergents, including, but not necessarily limited to, Triton X-100 (t-Octylphenoxypolyethoxyethanol) and its derivatives, Triton X-114, Triton X-405, Triton X-101, Triton N-42, Triton N-57, Triton N-60, Triton X-15, Triton X-35, Triton X-45, Triton X-102, Triton X-155, Triton X-165, Triton X-207, Triton X-305, Triton X-705-70 and Triton B-1956; Nonylphenyl Polyethylene Glycol (Nonidet P-40; NP-40, Igepal CA630); the Air Products series of Surfynol surfactants, including, but not necessarily limited to, Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, and Surfynol EP series, for example Surfynol 104 series (104, 104A, 104BC, 104DPM, 104E, 104H, 104NP, 104PA, 104PG50, 104S), and Surfynol 2502; Tyloxapol; n-Undecyl β-D-Glucopyranoside, and any non-ionic Octylphenol Ethoxylate surfactant. Additional non-limiting examples include the Dow Chemicals' Dowfax series of non-ionic surfactants, such as the N-series and the DP-series of surfactants, including, but not necessarily limited to, DOWFAX 63N10, DOWFAX 63N13, DOWFAX 63N30, DOWFAX 63N40, DOWFAX 81N13, DOWFAX 81N15, DOWFAX 92N20, DOWFAX 100N15, DOWFAX EM-51, DOWFAX 20A42, DOWFAX 20A64, DOWFAX 20A612, DOWFAX 206102, DOWFAX DF-101, DOWFAX DF-111, DOWFAX DF-112, DOWFAX DF-113, DOWFAX DF-114, DOWFAX DF-117, DOWFAX WP-310, DOWFAX 50C15, DOWFAX DF-121, DOWFAX DF-122, DOWFAX DF-133, DOWFAX DF-141, DOWFAX DF-142, and DOWFAX DF-161. Yet other additional non-limiting examples include the pluronic series of surfactants from BASF, including but not limited to, 10R5, 17R2, 17R4, 25R2, 25R4, 31R1, F108 series, F127 series, F38 series, F68 series, F77 series, F87 series, F88 series, F98 series, L10, L101, L121, L31, L34, L43, L44 series, L61, L62 series, L64, L81, L92, N-3, P103, P104, P105, P123, P65, P84, P85, and F127.

Any suitable amount of detergent and/or non-ionic surfactant may be included in the lysis buffer. In some embodiments, the lysis buffer comprises a non-ionic surfactant, such as Triton X-100, in an amount from about 0.05% to about 20%. In some embodiments, the lysis buffer comprises from about 0.05% to about 15%, from about 0.05% to about 10%, from about 0.05% to about 7%, from about 0.05% to about 5%, from about 0.05% to about 4%, from about 0.05% to about 3%, from about 0.05% to about 2%, from about 0.05% to about 1%, or from about 0.05% to about 0.5%. In some embodiments, the lysis buffer comprises from about 0.1% to about 15%, from about 0.1% to about 10%, from about 0.1% to about 7%, from about 0.1% to about 5%, from about 0.1% to about 4%, from about 0.1% to about 3%, from about 0.1% to about 2% or from about 0.1% to about 1%. In some embodiments, the lysis buffer comprises from about 0.5% to about 15%, from about 0.5% to about 10%, from about 0.5% to about 7%, from about 0.5% to about 5%, from about 0.5% to about 4%, from about 0.5% to about 3%, or from about 0.5% to about 2%. In some embodiments, the lysis buffer comprises from about 1% to about 15%, from about 1% to about 10%, from about 1% to about 7%, from about 1% to about 5%, from about 1% to about 4%, from about 1% to about 3%, or from about 1% to about 2% of a non-ionic surfactant, such as Triton X-100. In some embodiments, the lysis buffer comprises from about 2% to about 15%, from about 2% to about 10%, from about 2% to about 7%, from about 2% to about 5%, from about 2% to about 4%, or from about 2% to about 3% of a non-ionic surfactant, such as Triton X-100. In some embodiments, the lysis buffer comprises from about 3% to about 15%, from about 3% to about 10%, from about 3% to about 7%, from about 3% to about 5%, or from about 3% to about 4% of a non-ionic surfactant, such as Triton X-100. In some embodiments, the lysis buffer comprises about 0.05%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12% about 13%, about 14%, or about 15% a non-ionic surfactant, such as Triton X-100.

Although the lysis buffer may comprise one or more other components, and those components are not limited by the exemplary components disclosed herein, the lysis buffer will typically contain a solvent, such as water, an organic solvent, such as glycerol, or both. Although it is preferred that the solvent used be as pure as possible or practicable, solvents of any purity may be used. Thus, where water is included in the lysis buffer, it may be distilled water, double-distilled water, de-ionized water, sterilized water, or any combination thereof. The solvent, be it water or any other solvent or combination of water and any other solvent, may be treated before use to reduce or eliminate one or more chemical or biochemical activities, such as, but not limited to nuclease (e.g., RNase, DNase) activities. For example, the water or any other solvent or combination of water and any other solvent may be RNase-free. Likewise, the lysis buffer may be treated with sterilization techniques or with chemicals or biologicals, etc. to sterilize the composition or to reduce or eliminate one or more undesirable chemical or biochemical activities (e.g., RNase, DNase, etc.). For example, the lysis buffer may contain an RNase inhibitor.

The lysis buffer of the invention may also comprise one or more salts, such as a sodium salt, a potassium salt, a magnesium salt, a manganese salt, a zinc salt, a cobalt salt, or a combination of two or more of these salts. Specific exemplary salts include disodium phosphate, sodium chloride, magnesium chloride, manganese chloride, and potassium chloride. The salts may be added in any suitable amount and for any reason, including, but not limited to, as an aid in lysis of cells or viruses, for moderation of surfactant cloud point and foam level, and for improved function of reagents involved in amplification of nucleic acids.

The lysis buffer of the invention may also comprise one or more buffers suitable for inclusion in nucleic acid amplification, such as, but not limited to Tris-HCl.

The lysis buffer of the invention may also comprise one or more reducing agents. Reducing agents help to break bonds (such as disulfide bonds) which loosen the secondary structure of RNA and can facilitate reverse transcriptase (RT) enzyme initiation of transcription. Any suitable reducing agent may be included in the lysis buffer, such as dithiothreitol (DTT).

Without wishing to be bound by theory, the lysis buffer of the invention presumably is necessary for lysis of both infected cells (such as epithelial cells) as well as the virus. Any suitable lysis buffer can be used provided it does not interfere with the subsequent PCR reactions, such as RT-PCR.

The pH of the lysis buffer may be any suitable pH. In some embodiments, the pH of the lysis buffer is from about 6.5 to about 8.5 or from about 7.0 to about 8.5. For example, the pH of the lysis buffer can be from about 7.5 to about 8.5 or from about 7.5 to about 8.0. In some embodiments, the pH of the lysis buffer is about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.

The lysis buffer of the invention may be provided as a concentrated stock solution. For example, concentrated stock solutions can be formulated as a 100× stock, 50× stock, 20× stock, 10× stock, 5× stock, or 2× stock.

The lysis buffer of the invention may be made in advance, and optionally diluted before use. The lysis buffer may be diluted in any suitable diluent that does not interfere with the subsequent RT-PCR. In some embodiments, the lysis buffer is diluted with water before use. In other embodiments, the lysis buffer is diluted with a medium, such as, for example, a transport/storage medium. The lysis buffer can be diluted in any suitable ratio. For example, the lysis buffer may be diluted 100:1, 50:1, 25:1, 20:1, 10:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:40, 1:50, or 1:100 (lysis buffer:diluent).

In an aspect, the lysis buffer of the invention may be a concentrated stock comprising 1-5% Triton X-100 and one or more of the following components: 5-20% glycerol, 0.5-4 mM DTT, 10-50 mM Na2HPO4, and 10-50 mM Tris-HCl. In some embodiments, the lysis buffer of the invention may be a concentrated stock comprising about 3% Triton X-100 and one or more of the following components: about 10% glycerol, about 2 mM DTT, about 25 mM Na2HPO4, and about 25 mM Tris-HCl. The pH of the concentrate stock may be from about 7.5 to about 8.0, preferably about 7.8.

The concentrated stock may be diluted with a diluent, such as RNase-free water, prior to use. In some embodiments, the concentrated stock may be diluted from 1:15 to 1:1 with RNase-free water prior to use. In some embodiments, the concentrated stock may be diluted 1:6 with RNase-free water prior to use.

In some aspects, RNase inhibitor to the lysis buffer is added prior to use. In some embodiments, the RNase inhibitor is added to the lysis buffer in an amount from about 1:100 to about 1:10000 prior to use. In some embodiments, the RNase inhibitor is added to the lysis buffer in an amount of about 1:1000 prior to use.

Methods

In an aspect, the present invention provides a method of lysing at least one cell or virus using the lysis buffer of the invention. The method comprises contacting at least one cell or virus with a composition of the invention for a sufficient amount of time to cause the cell or virus to lyse.

The cell can be any eukaryotic cell. The cell can be any cell of interest, including, but not limited to, mammalian cells, avian cells, amphibian cells, reptile cells, and insect cells. For example, the cell can be a human cell, a monkey cell, a rat cell, a mouse cell, a dog cell, a cat cell, a pig cell, a horse cell, a hamster cell, a rabbit cell, a frog cell, an insect cell, etc. In some embodiments, the cell is an epithelial cell.

By “at least one cell”, it is meant not only a single cell, but a single cell type. Thus, two or more cells can mean not only two or more cells of the same cell type, but one or more cell of two different cell types. Unless otherwise specifically noted, it is not relevant whether a population of a single cell type is present or a population of two or more cell types is present. Regardless, the methods of the invention (including those discussed below) will provide the stated effects.

Furthermore, the terms “at least one cell” and “a cell” are, unless otherwise noted, used interchangeably herein to define a single cell, a collection of a single type of cell, or a collection of multiple types of cells, at least one cell of each type being present.

Similarly, the terms “at least one virus” and “a virus” are, unless otherwise noted, used interchangeably herein to define a single virus, a collection of a single type of virus, or a collection of multiple types of viruses, at least one virus of each type being present.

In some embodiments, the at least one cell or at least one virus is present in a biological sample.

In some aspects, the biological sample is collected using a swab. For example, the swab can be used to collect a biological sample from any mucous membrane such as, nose, cheek, pharynx, or mouth. In an aspect, the swab may be used to collect a sample from any place the cells of interest, for example epithelial cells, are located. In a particular embodiment, the biological sample is a nasal swab. In another embodiment, the biological sample is a bodily fluid (nasopharygeal aspirate, sputum, saliva, urine, blood, etc.), excrements, lavage, etc.

The biological sample can be from any animal, preferably a mammal, more preferably a human.

Other types of samples, such as liquid samples and smears from surfaces, for example, can also be used in the method of the invention.

The sample can optionally be stored by any suitable means prior to lysis. The means for storage can vary depending on the type of sample and length of storage. For example, biological samples can be stored at room temperature (e.g., 22-25° C.), placed on ice, or refrigerated (e.g., 4° C.) for short-term storage or can be frozen for long-term storage. Frozen biological samples can be stored for example at −20° C., −80° C. or in liquid nitrogen.

In some embodiments, the sample, such as a biological sample, is placed in a transport or storage medium for storage at any suitable temperature for any suitable time.

The amount of time the sample, optionally comprising at least one cell, is contacted with the lysis buffer of the invention may vary and can be determined by one of skill in the art. In some embodiments, the amount of time the sample is contacted with the lysis buffer of the invention is an amount of time sufficient to cause lysis of at least one cell or virus. Due to the chemical nature of the lysis reaction, it is envisioned that the time can be quite short, such as 1 second or less. However, the time need not be so limited. Indeed, because the lysis buffer can be present during subsequent storage and/or amplification of nucleic acids, the lysis time can be relatively long. Suitable times can range from 1 second or less to minutes, hours, or days. Exemplary times for contact include, but are not limited to, 10 seconds, 15 seconds, 20 seconds, 25 seconds, 30 seconds 35 seconds, 40 seconds, 45 seconds, 50 seconds, 55 seconds, 1 minute, 90 seconds, 2 minutes, 5 minutes, 10 minutes, 20 minutes, 30 minutes or more.

In various embodiments, additional optional steps are included in the lysis method. It has been demonstrated that cell or viral lysis will occur without additional steps by use of the lysis buffer of the invention. However, to expedite lysis, the cells and/or viruses can be exposed to one or more mechanical disruption techniques. Any known mechanical disruption technique may be used, including, but not limited to, vortexing, repeated pipeting, inversion, shaking, and stirring. Thus, lysis can be accomplished, at least in part, by homogenizing. Depending on the ultimate use of the lysate, the mechanical techniques, when used, may be applied gently to minimize shearing stresses on the nucleic acids. In some embodiments, lysis can be accomplished, at least in part, through the action of biological or biochemical substances. For example, lysis can be accomplished in the presence of a proteinase, such as Proteinase K.

Any suitable volume of lysis buffer may be used in the method of the invention and can be varied depending on the sample. In some embodiments, a volume of about 50 μl, about 100 μl, about 200 μl or more of lysis buffer is used. In some embodiments, a volume of 100 μl of lysis buffer is used.

Freezing of the lysate can improve cell and/or virus lysis. Thus, in some embodiments, the lysate may optionally be frozen prior to analysis. The lysate may be frozen once or more than once. In other words, the lysate may be subjected to one or multiple freeze-thaw cycles. Any suitable conditions may be used to freeze the lysates. It will be understood that temperature and duration can be optimized depending on various factors, such as the size and type of the sample and amount of lysate. In some embodiments, the lysate is incubated at a temperature of −20° C. or less prior to analysis. For example, the lysate may be incubated at a temperature selected from, but not limited to, about −20° C., about −80° C., or about −120° C. prior to analysis. In some embodiments, the lysates are placed on dry ice prior to analysis. The lysate may be incubated as described above for any suitable amount of time. In some embodiments, the lysate may be incubated at a temperature of −20° C. or less for at least about 10 minutes. For example, the lysate may be incubated at a temperature of −20° C. or less for about 10 minutes, about 30 minutes, about 1 hour, or about 24 hours, or more. In some embodiments, the lysates are incubated at a temperature of −80° C. for 10 minutes prior to analysis.

Another optional step in the lysis method is storing the lysate for a period of time before use. In such embodiments, the lysate can be stored at any number of temperatures. For example, it can be stored at relatively high temperatures (e.g., 37° C.), at room temperature (e.g., 22° C.-25° C.), in the refrigerator (e.g., 4° C.), frozen (e.g., −20° C.), or deep frozen (e.g., −80° C. or lower).

The lysis method can also include one or more steps that result in separation of cell and/or viral components from other cell and/or viral components. Thus, for example, the method may comprise centrifuging the lysate to remove unlysed cells and/or viruses, cell and/or viral membranes and proteins from nucleic acids. While not preferred, it can also include precipitation of one or more cellular or viral component from others, for example, by the addition of one or more salts, organic solvents (e.g., alcohol), or through heat treatment and subsequent centrifugation. Other techniques for separating cellular and/or viral components from each other are known to those of skill in the art, and any suitable technique may be used, each being selected based on the desired outcome. Selection and performance of an appropriate technique is well within the skill level of those of skill in the art.

The lysis method may also include manipulation of one or more lysate component. Thus, the method may include purification of one or more nucleic acid from the lysate and/or amplification of one or more nucleic acid from the lysate. Various embodiments of the method of lysis include any and all procedures that are known for use with lysates.

In an embodiment, the method of lysing at least one cell and/or at least one virus comprises adding the lysis buffer of the invention to the at least one cell and/or at least one virus. In some embodiments, the method further comprises incubating the mixture of the lysis buffer and the at least one cell and/or the at least one virus for 5 minutes at room temperature.

In another embodiment, a method of lysing a sample is provided, the method comprising mixing the sample with the lysis buffer of the invention. In some embodiments, the sample is a biological sample. In some embodiments, the biological sample is a swab, such as a nasal swab. In some embodiments, the biological sample comprises at least one cell, such as an epithelial cell and/or at least one virus, such as a coronavirus. In some embodiments, the nasal swab is placed in 100 μl of the lysis buffer of the invention. In some embodiments, the method further comprises squeezing the nasal swab to release the sample into the lysis buffer. In some embodiments, the method further comprises incubating the mixture of the lysis buffer and the biological sample for 5 minutes at room temperature to produce a lysate. In some embodiments, the method further comprises centrifuging the lysate, for example at 12,000 rpm for 2 minutes at room temperature, and collecting the supernatant.

The present invention also provides a method for amplifying one or more nucleic acids. In general, the method comprises contacting a sample with the lysis buffer of the invention to produce a lysate as described above and amplifying at least one nucleic acid in the lysate.

Numerous techniques for amplification of nucleic acids are known and widely practiced in the art, and any of those techniques are applicable according to the method of this invention. One of skill in the art may select the amplification method based on any number of considerations, including, but not limited to, speed, sensitivity, usefulness in amplifying a particular type of nucleic acid (e.g., RNA vs. DNA), and reliability.

Although the method may comprise isolation or purification (to at least some extent) of nucleic acids, it has surprisingly been discovered that amplification of target nucleic acids may be accomplished without purification of the nucleic acid beforehand. Thus, the lysate of the invention is suitable for direct nucleic acid amplification. In some embodiments, amplifying is by a PCR technique. In certain embodiments, the PCR technique is qPCR or RT-PCR (including RT-qPCR).

Where one or more RNA are the nucleic acids of interest, the method may comprise a cDNA synthesis prior to, or at the time of, amplification. Numerous cDNA synthesis protocols are known in the art, and any suitable protocol may be used. For example, the qScript XLT reverse transcriptase from Quantabio may be used to prepare cDNA from RNA templates.

It has been shown that the methods of the invention are particularly well-suited for use to detect viral RNA or DNA within mammalian biological samples, such as a nasal swab, or non-biological samples.

The method of the invention provides several advantages over conventional viral RNA or DNA detection assays. For example, the method of the invention avoids the need for an RNA extraction step, thereby reducing labor and making the method of the invention faster. RNA extraction kits, such as QIAamp® DSP Viral RNA Mini kit (Qiagen) which is currently required by the Centers for Disease Control and Prevention (CDC) in the United States for COVID-19 testing, generally contain columns to separate and purify nucleic acids from components that may interfere with amplification methods. Therefore, the elimination of an RNA extraction step also results is less material consumption since no columns are required. Furthermore, reagents necessary for RNA extraction are not always readily available, thus, elimination of these reagents increases availability of the assays. This can be of particular importance during pandemics, for example, when a high volume of test samples need to be analysed and rapid results obtained. The provision of a high-throughput, rapid method to detect viral RNA that is both precise and sensitive is a significant advantage.

The method of the invention preferably comprises incubating a biological or non-biological sample (e.g., a nasal swab) with a lysis buffer and using the lysate directly for RT-PCR (e.g., one-step RT-qPCR). The method of the invention preferably is performed without an RNA or DNA extraction step.

In some embodiments, one or more control reactions are included, such as a control reaction to permit normalization of the amount of nucleic acid being amplified with respect to other amplification reactions that are being performed concurrently, or with respect to a standard amplification curve. Such control reactions can comprise adding one or more exogenous nucleic acids to the reaction and performing an amplification on that nucleic acid. The control reaction can alternatively comprise amplifying sequences present in nucleic acids naturally present in the cell or biological sample of interest, where such sequences have a known copy number and amplification efficiency. In other embodiments, control reactions for known sequences are performed in reaction vessels separate from the reaction vessel in which the amplification of interest is being performed. Various other control reactions are known and widely used for amplification reactions, and any of those control reactions may be included in the method of the present invention to determine the success and efficiency of one or more steps in the amplification process.

In some embodiments, the control reaction is an internal control that permits the practitioner to evaluate, and thus normalize if desired, the number of cells present in a particular cell lysate sample. In this way, conclusions about the amount of target nucleic acid (e.g., a particular viral RNA or DNA gene) in a sample may be made. More specifically, when comparing amplification results of two different samples, it is often not possible to determine with a high degree of accuracy, the number of cells or virus in the original sample, the number of cells or virus successfully lysed, or the total amount of nucleic acid liberated from each sample. Thus, accurate comparisons of the total amount of a target nucleic acid in different samples is not possible. Currently, housekeeping genes or rRNA species are used as markers to standardize or normalize samples from different cells or tissues. However, the currently used internal standards have been reported to be inconsistent, and thus do not provide the accuracy and repeatability that is needed for an internal control.

An internal control that is standardized among different samples and cell types is thus a desirable feature of a PCR protocol. In certain embodiments, the present invention encompasses such an internal control by including within the compositions, methods, and kits, primers that are specific for unique sequences on one or more of the chromosomes of a given cell or virus. Because these unique genomic sequences are present in only one copy per haploid genome (i.e., two copies per cell), they can be used to prepare a standard curve for a particular amplification procedure. Inclusion of the primers in the amplification reactions for test samples (either in the same reaction vessel or in a second reaction vessel comprising the same components) results in an amplification curve for the unique genomic sequences. These curves can be compared to the standard curve for each primer set, and the amount of nucleic acid, and thus the number of cells or viruses in the original sample, can be calculated. With this knowledge, the amount of a target nucleic acid in numerous different samples can be determined, and accurately compared with other samples.

In some embodiments, the method of the invention can be used to detect an RNA or DNA virus using the lysis buffer described herein. For example, the method of the invention can be used to detect an RNA or DNA virus such as, but not limited to, togavirus, coronavirus, retrovirus, picornavirus, calicivirus, reovirus, orthomyxovirus, paramyxovirus, rhabdovirus, buynavirus, arenavirus, or fibovirus. In some embodiments, the RNA virus is a coronavirus such as, but not limited to, corona SARS viruses, such as SARS CoV or SARS CoV-2 or variants of those viruses. In a particular embodiment, the method of the invention can be used to detect SARS CoV-2.

Thus, the present invention also relates to a method for the identification of a subject infected with SARS CoV-2 comprising obtaining a lysate from a biological sample obtained from the subject, reverse transcribing the RNA within the lysate to obtain cDNA, and subjecting the cDNA to PCR assay using a set of primers derived from a nucleotide sequence of the SARS CoV-2 genome.

Primers

Any suitable primers can be used in the methods of the invention. Suitable primers can readily be identified by those of skill in the art depending upon the target nucleic acid of interest to be identified. Several primers useful to identify common nucleic acids of interest are commercially available and can be used in the methods of the invention.

In some embodiments, the primers are directed to an RNA viral genome, such as the SARS CoV-2 genome or to the genome of SARS CoV-2 variants. The complete SARS CoV-2 genomic sequence can be found in GenBank Accession No. MN908947.3. Kits for the detection of SARS CoV containing primers are commercially available from various vendors. It is contemplated that such primers can be used in the method of the invention. In some embodiments, the primers are directed to the E gene, the N gene, or the RdRP gene. In some embodiments, the primers are directed to the E gene and comprise or consist of SEQ ID NO: 8 and/or SEQ ID NO: 9, or a complement thereof. In some embodiments, the primers are directed to the RdRP gene and comprise or consist of SEQ ID NO: 11 and/or SEQ ID NO: 12, or a complement thereof.

Probes

Any suitable nucleic acid probes can be used in the method of the invention. Suitable nucleic acid probes can readily be identified by those of skill in the art depending upon the target nucleic acid of interest to be identified. Several probes useful to identify common nucleic acids of interest are commercially available and can be used in the methods of the invention.

In some embodiments, the nucleic acid probes are directed to an RNA or DNA viral genome, such as the SARS CoV-2 genome the genome of a SARS CoV-2 variant. Kits for the detection of SARS CoV containing nucleic acid probes are commercially available from various vendors. It is contemplated that such probes can be used in the method of the invention. In some embodiments, the nucleic acid probes are directed to the E gene, the N gene, or the RdRP gene. In some embodiments, the nucleic acid probe is directed to the E gene and comprises or consists of SEQ ID NO: 7, or a complement thereof. In some embodiments, the nucleic acid probe is directed to the RdRP gene and comprises or consists of SEQ ID NO: 10, or a complement thereof.

In some embodiments, the probes can be tagged at 5′ and/or 3′ end, for instance with a fluorophore and/or a quencher. The tag can be any suitable tag, such as a HEX tag or a BHQ1 tag.

The RT-qPCR protocol described herein is based on standard diagnostic TaqMan-based RT-qPCR methods for CoV-2. In principle, the protocol can be varied, such as, for example, other viral genes could be measured, different primer and probe sequences, different master mixes, quenchers, fluorophores, apparatuses, etc could be used. Other RT-qPCR methods such as sybr green based methods can also be used in the method of the invention.

Kits are also provided. In general, the kits contain the lysis buffer of the invention. The kits can further comprise one or more substances, materials, reagents, etc. that can be used for lysis of cells or viruses, storage of nucleic acids or cell lysates, or manipulation or analysis of nucleic acids, such as amplification of nucleic acids. In some embodiments, some or all of the materials, reagents, etc., necessary to lyse cells or viruses, amplify nucleic acids, and/or purify nucleic acids are included in the kit.

For example, a kit may contain a container holding the lysis buffer of the invention, and, in the same or a separate container, at least one reagent for amplification of a target nucleic acid. Thus, it can comprise at least one primer, such as two primers, for amplification of a target nucleic acid. It also may include at least one other primer for amplification of a target nucleic acid, which can be, but is not necessarily, the same nucleic acid (and even the same sequence within the same nucleic acid) that is the target for one or more other primer(s) in the kit. In some embodiments, the kits comprise two or more primers for amplifying one or more unique genomic sequences.

A kit of the present invention may be a kit for analysis of nucleic acids, further comprising a lysis buffer of the invention. For example, it can be a kit for detection of RNA using a one-step RT-qPCR, further comprising at least one container containing the lysis buffer of the invention. Such kits can comprise, in packaged combination, at least one reverse transcriptase, at least one DNA polymerase, such as Taq DNA polymerase, Pfu DNA polymerase, Pfx DNA polymerase, Tli DNA polymerase, Tfl DNA polymerase, and klenow, an RNase inhibitor, nucleotides (e.g., any or all of the four common deoxynucleotides), primers, probes, or labels (such as, for example, SYBR green), or any combination of two or more of these. Alternatively, it can be a kit that can be used for detection of RNA using a two-step RT-qPCR. Alternatively, the kit can be one that can be used for detection of DNA using a PCR technique, such as qPCR. In addition, the kit can be one that is used for detection of short interfering RNA (siRNA). In some embodiments, the kits comprise transfection or transformation reagents.

The kits can comprise the components in a single package or in more than one package within the same kit. Where more than one package is included within a kit, each package can independently contain a single component or multiple components, in any suitable combination. As used herein, a combination of two or more packages or containers in a single kit is referred to as “in packaged combination”. The kits and containers within the kits can be fabricated with any known material. For example, the kits themselves can be made of a plastic material or cardboard. The containers that hold the components can be, for example, a plastic material or glass. Different containers within one kit can be made of different materials. In embodiments, the kit can contain another kit within it. For example, the kit of the invention can comprise a kit for purifying nucleic acids.

The kit of the invention can comprise one or more components useful for amplifying target sequences. In embodiments, some or all of the reagents and supplies necessary for performing PCR are included in the kit. In some embodiments, some or all reagents and supplies for performing qPCR are included in the kit. In other embodiments, some or all reagents and supplies for performing RT-PCR are included in the kit. Non-limiting examples of reagents are buffers (e.g., a buffer containing Tris, HEPES, and the like), salts, and a template dependent nucleic acid extending enzyme (such as a thermostable enzyme, such as Taq polymerase), a buffer suitable for activity of the enzyme, and additional reagents needed by the enzyme, such as dNTPs, dUTP, and/or a UDG enzyme. A non-limiting example of supplies is reaction vessels (e.g., microfuge tubes).

The kit can comprise at least one dye for detecting nucleic acids, including, but not limited to, dsDNA. In embodiments, the kit comprises a sequence-non-specific dye that detects dsDNA, such as SYBR® Green dye (Molecular Probes, Eugene, Oreg.). The dye is preferably contained alone in a container. In embodiments, the dye is provided as a concentrated stock solution, for example, as a 50× solution. In embodiments, the kit comprises a passive reference dye. In these embodiments, the passive reference dye can be included in the kit alone in a separate container. The passive reference dye can be provided as a concentrated stock solution, for example, as a 1 mM stock solution. A non-exclusive exemplary passive reference dye is ROX dye. In embodiments, the kit contains either a DNA-detecting dye or a passive reference dye. In other embodiments, the kit contains both a DNA-detecting dye and a passive reference dye.

The invention, in general, is suitable for use in both research and diagnostics. That is, the compositions and methods of the invention can be used for the purpose of identifying various nucleic acids or expressed genes, or for other research purposes. Likewise, the compositions and methods can be used to diagnose numerous diseases or disorders of humans and animals. In addition, they can be used to identify diseased or otherwise tainted food products (e.g., foods that are infected with one or more pathogenic organisms), or the presence of toxic substances or toxin-producing organisms in a sample. Thus, the compositions and methods have human health and veterinary applications, as well as food testing and homeland security applications.

The present invention may be better understood by reference to the following examples, which are not intended to limit the scope of the claims.

### EXAMPLES

Materials and Methods

Nasal swabs were collected using craft cotton swabs (Cat. no. 87104**260; Tamiya) and transferred to a 1.5 ml reaction tube or deep well microtiter plate with 100 μl lysis buffer (3% Triton X-100, 10% Glycerin, 2 mM DTT, 25 mM Na2HPO4, 25 mM TRIS HCL; pH 7.8). The lysis buffer was stored at RT and diluted 1:6 with RNase-free water (Invitrogen, Ultrapure distilled Water Dnase/Rnase free, 10977-035) prior to use. On day of usage: 0.1 μl RNAse Inhibitor (NEB, M0314L, 40 U/μl) per 100 μl sample lysis volume.

After the swab was placed in the lysis buffer, the swab was compressed to release the sample into the lysis buffer and incubated for 5 minutes. The sample was then kept on ice for direct use or frozen at −20° C. (short-term) or −80° C. (long-term storage).

Before use, the sample was thawed (if necessary) and centrifuged at 12,000 rpm for 2 minutes at RT. Following centrifugation, the supernatant was transferred to a new reaction tube or deep well microtiter plate. The samples can optionally be frozen at this stage before proceeding with real-time one-step RT-PCR.

For the real-time One-step RT-PCR assay, 2 μl lysate was transferred to a 384-well QPCR microplate (LightCycler® 480 Multiwell Plate 384 white, 4 barcodes No.: 05217555001) using a 10 μl multipipette or an automated pipetting system (e.g., Cybi Well Vario, Analytik Jena). Eight μl PCR MasterMix (Quantabio, UltraPlex™ 1-step ToughMix®, No.: 95166-01K) was added using a 100 μl multipipette or dispenser. The microplate was then sealed using clear qPCR foil (MicroAmp™ Optical Adhesive Film, ThermoScientific, 4311971) manually or with a heat sealer, and centrifuged at 1500 rpm for 3 minutes. The One-step RT-PCR was performed using LC480 (Roche, 384 well) and the data were analysed with LC480 software. The cycle threshold (CP or Cp) was calculated for each reaction and represents the number of cycles during the PCR amplification until the measured fluorescence reaches a value where the fluorescence can be distinguished from the background fluorescence.

Probes/Primers:
CLDN1 (Gene ID: 9076; claudin 1, human
epithelial cell marker)
CLDN1_Probe:
(SEQ ID NO: 1)
5′_HEX-CAGGCTCTCTTCACTGGCTGGGC-BHQ1_3′
CLDN1_F1 (forward primer):
(SEQ ID NO: 2)
5′-CCAGTCAATGCCAGGTACGA-3′
CLDN1_R1 (reverse primer):
(SEQ ID NO: 3)
5′-GAAGGCAGAGAGAAGCAGCA-3′
RPL32 (Gene ID: 6161; ribosomal protein L32;
human housekeeping gene)
RPL32_Probe:
(SEQ ID NO: 4)
5′_HEX-AATTAAGCGTAACTGGCGGAAACCC-BHQ1_3′
RPL32_F1 (forward primer):
(SEQ ID NO: 5)
5′-GCACCAGTCAGACCGATATGT-3′
RPL32_R1 (reverse primer):
(SEQ ID NO: 6)
5′-ACCCTGTTGTCAATGCCTCT-3′
E Gene Sarbeco (E gene, SARS CoV2 gene)
E_Gene Sarbeco_P1 (probe):
(SEQ ID NO: 7)
5′-Hex-ACACTAGCCATCCTTACTGCGCTTCG-BHQ-1-3′
E_Gene Sarbeco_F1 (forward primer):
(SEQ ID NO: 8)
5′-ACAGGTACGTTAATAGTTAATAGCGT-3′
E_Gene Sarbeco_R2 (reverse primer):
(SEQ ID NO: 9)
5′-ATATTGCAGCAGTACGCACACA-3′
RdRP_SARSr (RdRP SARS CoV2 gene; specific for
SARS CoV2, will not detect
SARS CoV)
RdRP_SARSr-P2 (probe):
(SEQ ID NO: 10)
5′-Hex-CAGGTGGAACCTCATCAGGAGATGC-BHQ-1-3′
RdRP_SARSr-F2 (forward primer):
(SEQ ID NO: 11)
5′-GTGARATGGTCATGTGTGGCGG-3′
RdRP_SARSr-R1 (reverse primer):
(SEQ ID NO: 12)
5′- CARATGTTAAASACACTATTAGCATA-3′

### Example 1—Detection of Epithelial Cells

RT qPCR for the epithelial marker CLDN1 and the human housekeeping gene RPL32 was performed according to the described protocol. Briefly, nasal swabs from ten healthy human volunteers were collected and lysed as described in the Materials and Methods. The lysates were directly used for RT qPCR without freezing. Control samples included swab only (swab without sample) and NTC (no template control).

CLDN1 and RPL32 probes and primers were included such that the final concentration of each oligo in the reaction was 333 nM.

PCR Reaction setup: 2.5 μl UltraPlex 1-step tough mix (4×), 2.5 μl primer/probe mix, 2.0 μl lysed nasal sample aliquot, 3.0 μl RNase-free water.

PCR Protocol: Reverse transcription was performed for 10 minutes at 50° C., followed by denaturation for 5 minutes at 95° C. 45 cycles: 1 min 95° C.+30 sec 60° C., or, more preferably, 15 sec 95° C.+1 min 60° C. After cycling, cooling was performed for 30 seconds at 40° C.

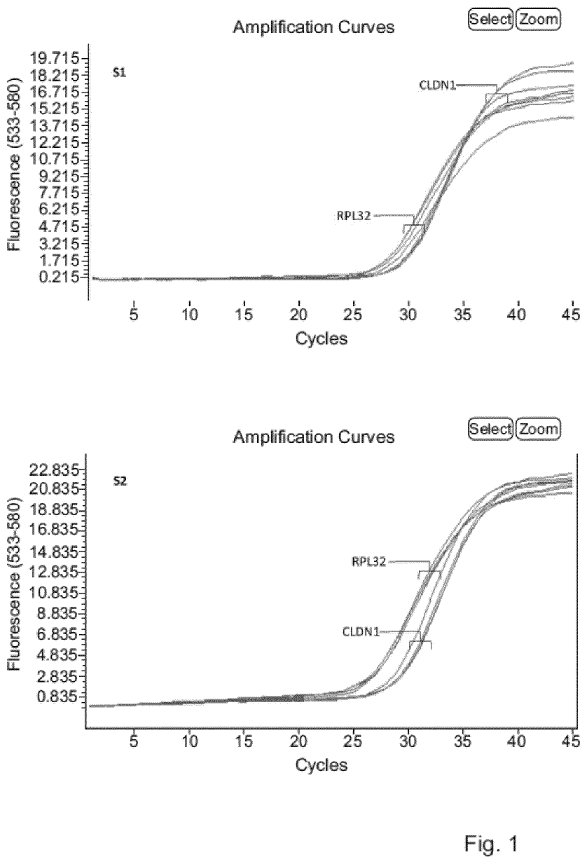

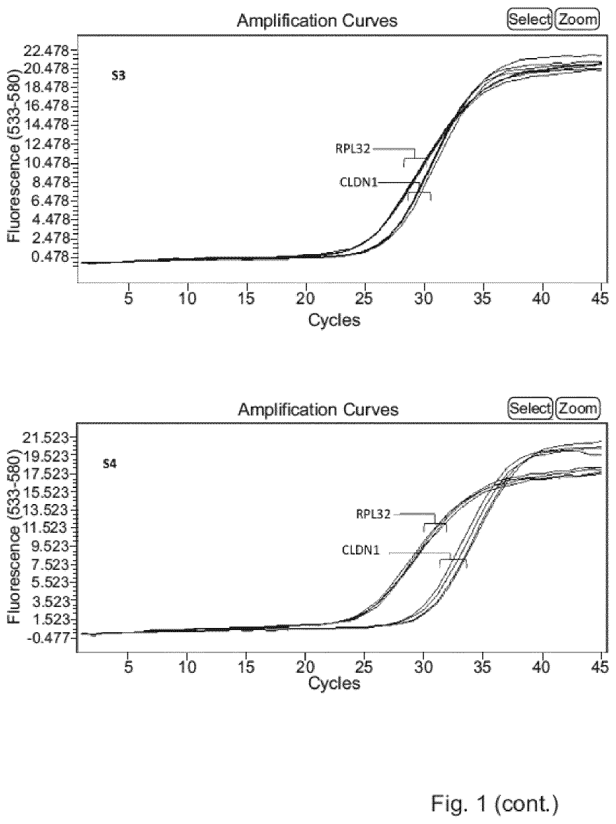

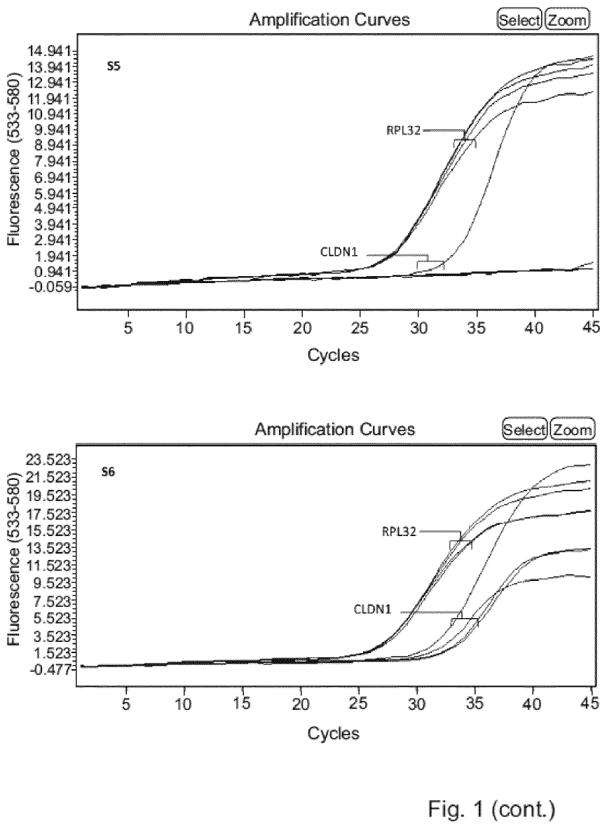

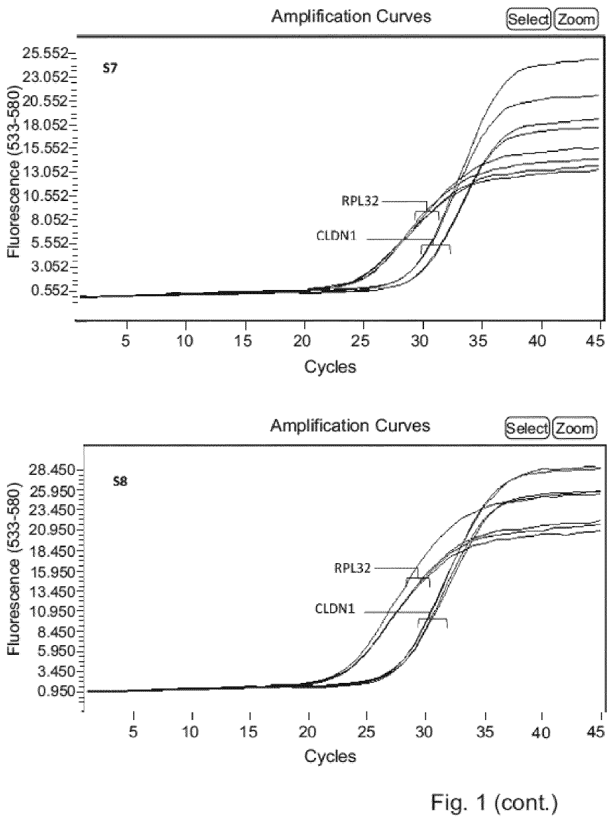

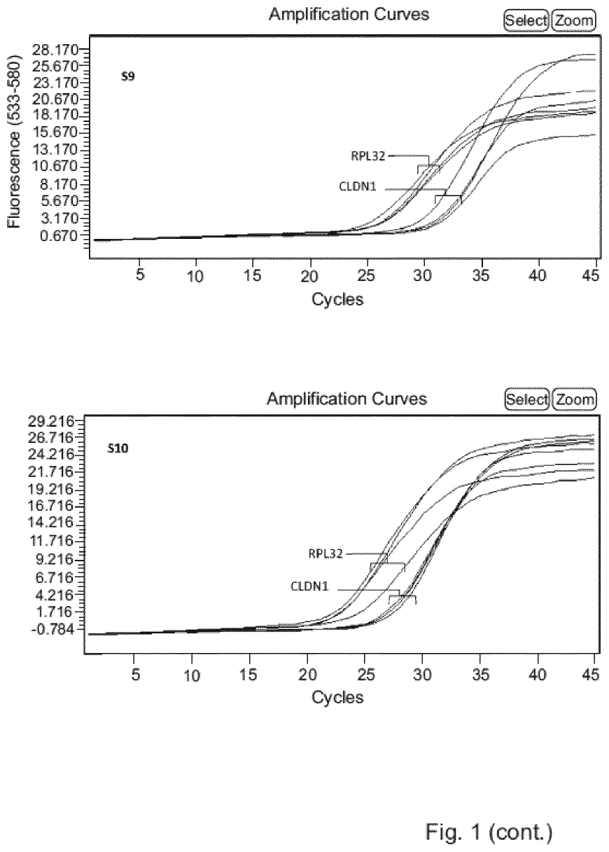

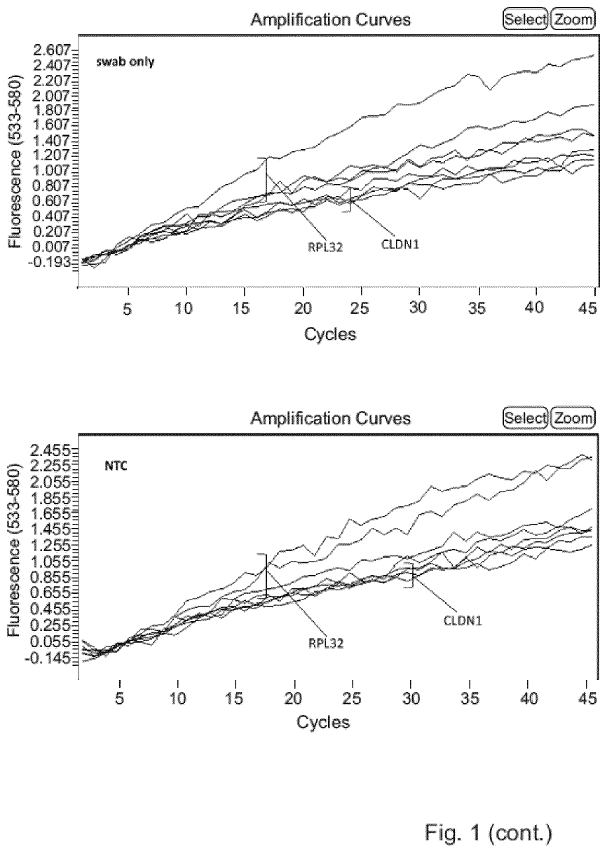

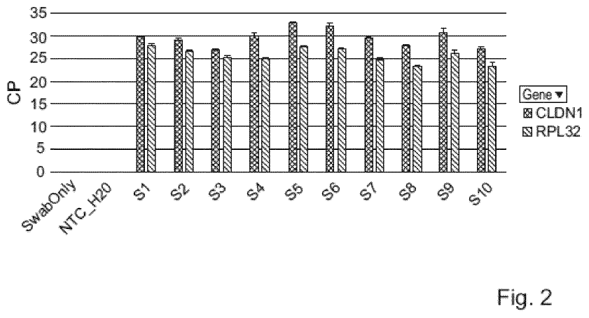

Amplification curves from each sample are provided in FIG. 1 and summarized in FIG. 2. These data illustrate that genes can be reliably detected from nasal swab samples prepared without inclusion of an RNA extraction step. Variability in detection level can be attributed to individual sampling.

### Example 2—Sensitivity of Viral Gene Detection by RT qPCR

The RT-qPCR protocol used was as described in TIB MOLBIOL/Roche Kit manual with the following exceptions: an 8 minute RT step was used. Optionally, UltraPlex™ 1-step ToughMix® was used instead of Roche RNA Virus Master Mix. The protocol is provided below in Table 1.

TABLE 1
Program Step:

| Parameter | RT Step | Denaturation | Cycling | | | Cooling |
|---|---|---|---|---|---|---|
| Analysis Mode | None | None | Quantification Mode | | | None |
| Cycles | 1 | 1 | 45 | | | 1 |
| Target [° C.] | 55 | 95 | 95 | 60 | 72 | 40 |
| Hold [hh:mm:ss] | 00:08:00 | 00:05:00 | 00:00:05 | 00:00:15 | 00:00:15 | 00:00:30 |
| Ramp Rate | 4.6 | 4.6 | 4.6 | 2.4 | 4.6 | 2.0 |
| [° C./s] | | | | | | |
| Acquisition Mode | None | None | None | Single | None | None |

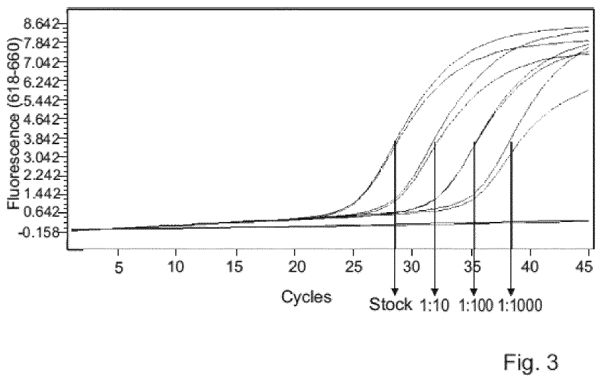

The sensitivity of RT qPCR for detection of a viral positive control commonly used in virus RNA extraction kits (LightMix® Modular EAV RNA Extraction Control, TIB MOLBIOL Cat. No. 58-0909-96) was tested. This was a typical RT qPCR reaction where the viral template RNA was added at various dilutions (no dilution, 1:10, 1:100, 1:1000) to the PCR mix. No lysate was used. The sensitivity of the RT qPCR for this gene is tested. Amplification curves are shown in FIG. 3 and data are provided below in Table 2.

TABLE 2

| Pos | Cp | Concentration | Mean CP | delta CP |
|---|---|---|---|---|
| I3 | 24.94 | 1x Stock | 25.01 | |
| I4 | 25.08 | 1x Stock | | |
| I5 | 28.22 | 1/10 | 28.27 | 3.26 |
| I6 | 28.32 | 1/10 | | |
| I7 | 31.78 | 1/100 | 31.83 | 3.56 |
| I8 | 31.88 | 1/100 | | |
| I9 | 34.73 | 1/1000 | 34.84 | 3.01 |
| I10 | 34.95 | 1/1000 | | |
| I11 | | H2O | | |
| I12 | | H2O | | |

The sensitivity of RT qPCR for detection of three CoV-2 genes: E-gene (LightMix® Modular SARS and Wuhan CoV E-gene, TIB MOLBIOL Cat. No. 53-0776-96), N-gene (LightMix® Modular SARS and Wuhan CoV N-gene, TIB MOLBIOL Cat. No. 53-0775-96), and RdRP-gene (LightMix® Modular Wuhan CoV RdRP-gene, TIB MOLBIOL Cat. No. 53-0777-96) were performed using the same protocol as described above for detection of EAV positive control. Various concentrations of each gene were included in the PCR mix (2× stock, 1× stock, 1:10 dilution, 1:100 dilution), corresponding to approximately 1000 copies (2×), 500 copies (1×), 50 copies (1:10) and 5 copies (1:100) tested.

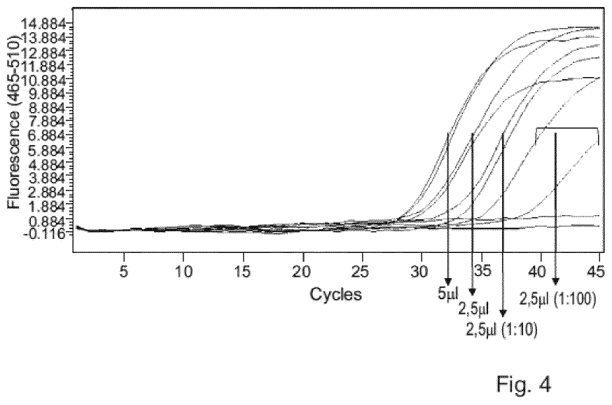

Amplification curves for detection of E-gene are shown in FIG. 4 and data are provided below in Table 3.

TABLE 3

| Pos | Cp | Concentration | Copy number | Mean CP | delta CP |
|---|---|---|---|---|---|
| I3 | 28.69 | 2x Stock | 1000 | 28.87 | |
| I4 | 29.05 | 2x Stock | 1000 | | |
| I5 | 30.29 | 1x Stock | 500 | 30.39 | 1.52 |
| I6 | 30.49 | 1x Stock | 500 | | |
| I7 | 33.46 | 1/10 | 50 | 33.30 | 2.91 |
| I8 | 33.14 | 1/10 | 50 | | |
| I9 | 35.16 | 1/100 | 5 | 37.04 | 3.74 |
| I10 | 38.91 | 1/100 | 5 | | |
| I11 | | H2O | 0 | | |
| I12 | | H2O | 0 | | |

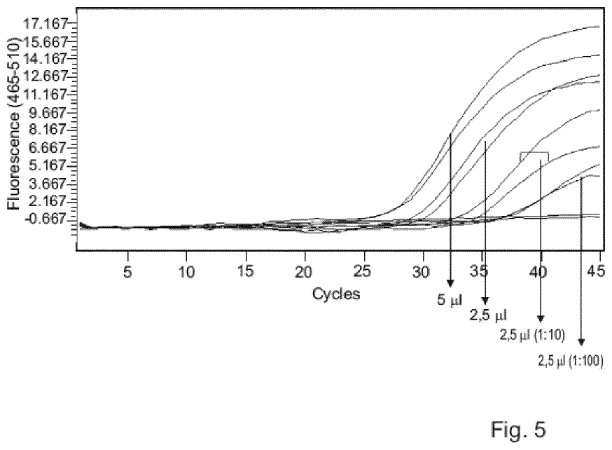

Amplification curves for detection of N-gene are shown in FIG. 5 and data are provided below in Table 4.

TABLE 4

| Pos | Cp | Concentration | Copy Number | Mean CP | delta CP |
|---|---|---|---|---|---|
| I3 | 28.12 | 2x Stock | 1000 | 28.10 | |
| I4 | 28.08 | 2x Stock | 1000 | | |
| I5 | 29.66 | 1x Stock | 500 | 30.01 | 1.91 |
| I6 | 30.35 | 1x Stock | 500 | | |
| I7 | 33.92 | 1/10 | 50 | 33.81 | 3.80 |
| I8 | 33.69 | 1/10 | 50 | | |
| I9 | 36.96 | 1/100 | 5 | 36.71 | 2.91 |
| I10 | 36.46 | 1/100 | 5 | | |
| I11 | | H2O | 0 | | |
| I12 | | H2O | 0 | | |

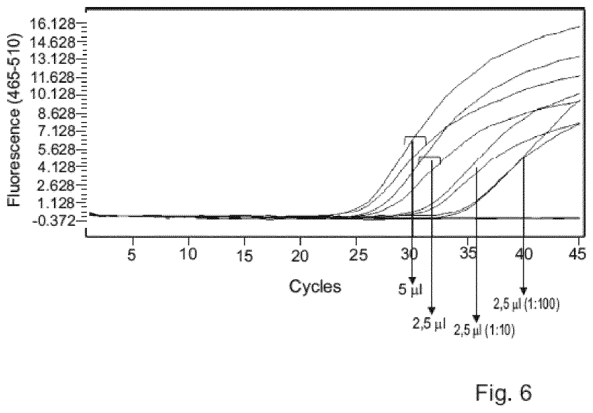

Amplification curves for detection of the RdRP-gene which is specific for detection of SARS CoV-2 are shown in FIG. 6 and data are provided below in Table 5.

TABLE 5

| Pos | Cp | Concentration | Copy Number | Mean CP | delta CP |
|---|---|---|---|---|---|
| I3 | 25.65 | 2x Stock | 1000 | 25.63 | |
| I4 | 25.61 | 2x Stock | 1000 | | |
| I5 | 27.66 | 1x Stock | 500 | 27.34 | 1.71 |
| I6 | 27.01 | 1x Stock | 500 | | |
| I7 | 31.37 | 1/10 | 50 | 31.32 | 3.99 |
| I8 | 31.27 | 1/10 | 50 | | |
| I9 | 34.39 | 1/100 | 5 | 34.56 | 3.24 |
| I10 | 34.73 | 1/100 | 5 | | |
| I11 | | H2O | 0 | | |
| I12 | | H2O | 0 | | |

### Example 3— Detection of CoV-2 Genes in Nasal Swab Lysates

RT qPCR for two of the viral genes tested in Example 2 (E-gene and RdRP gene) was performed according to the described protocol. Briefly, nasal swabs from ten healthy human volunteers (S1-S10) were collected and lysed as described in the Materials and Methods, to which CoV-2 RNA templates (TibMolBiol) were added (spiked). The lysates were directly used for RT qPCR without freezing.

For each sample S1-S10, the following reactions were performed: 1) E-Gene_Sample (no CoV-2 RNA template added; E-Gene probe/primers included); 2) E-Gene_SpikeRNA (CoV-2 RNA template added; E-Gene probe/primers included); 3) E-Gene_PosCntIRNA (CoV-2 RNA template only; E-Gene probe/primers included); 4) NTC_E-Gene (no template negative control); 5) RdRP-Gene (no CoV-2 RNA template added; RdRP probe/primers included); 6) RdRP-Gene_SpikeRNA (CoV-2 RNA template added; RdRP-Gene probe/primers included); 7) RdRP-Gene_PosCntIRNA (CoV-2 RNA template only; RdRP-Gene probe/primers included); and 8) NTC_RdRP-Gene (no template negative control). Each reaction was performed in quadruplicate.

E-gene probes and primers were included at the following final concentrations: E_Gene Sarbeco_P1 probe 400 nM, E_Gene Sarbeco_F1 forward primer 400 nM, E_Gene Sarbeco_R2 reverse primer 200 nM or, more preferably, E_Gene Sarbeco_P1 probe 200 nM, E_Gene Sarbeco_F1 forward primer 400 nM, E_Gene Sarbeco_R2 reverse primer 400 nM.

RdRP probes and primers were included at the following final concentrations: RdRP_SARSr-P2 probe 600 nM, RdRP_SARSr-F2 forward primer 800 nM, RdRP_SARSr-R1 reverse primer 100 nM, or, more preferably, RdRP_SARSr-P2 probe 100 nM, RdRP_SARSr-F2 forward primer 600 nM, RdRP_SARSr-R1 reverse primer 800 nM.

PCR Reaction setup: 2.5 μl UltraPlex 1-step tough mix (4×), 2.5 μl primer/probe mix (4×), 0.25 μl Positive control RNA from LightMix kit, 2.0 μl lysed nasal sample aliquot, 2.75 μl RNase-free water. In samples 1) and 5), no Positive control RNA was added and 3.0 μl RNase-free water was added.

PCR Protocol: Reverse transcription was performed for 10 minutes at 50° C., followed by denaturation for 5 minutes at 95° C. 45 cycles: 1 min 95° C.+30 seconds 60° C., or, more preferably, 15 sec 95° C.+60 seconds 60° C. After cycling, cooling was performed for 30 seconds at 40° C.

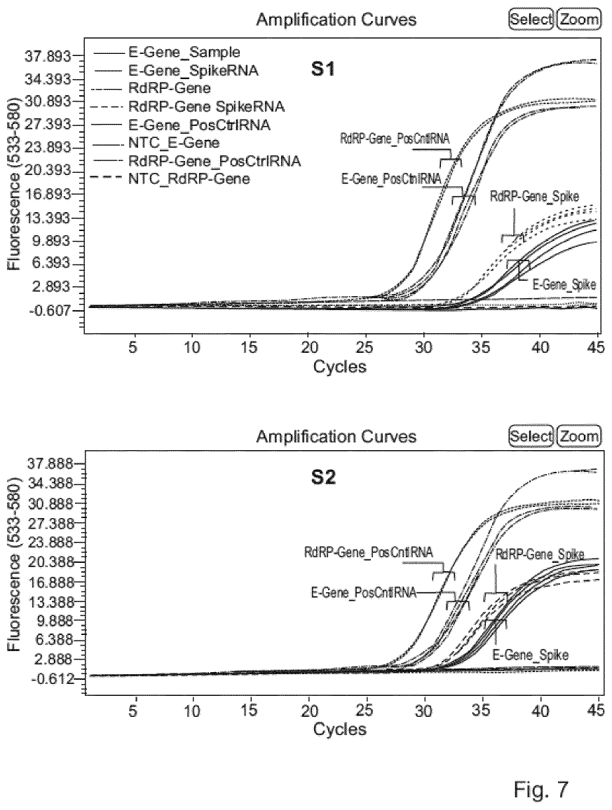

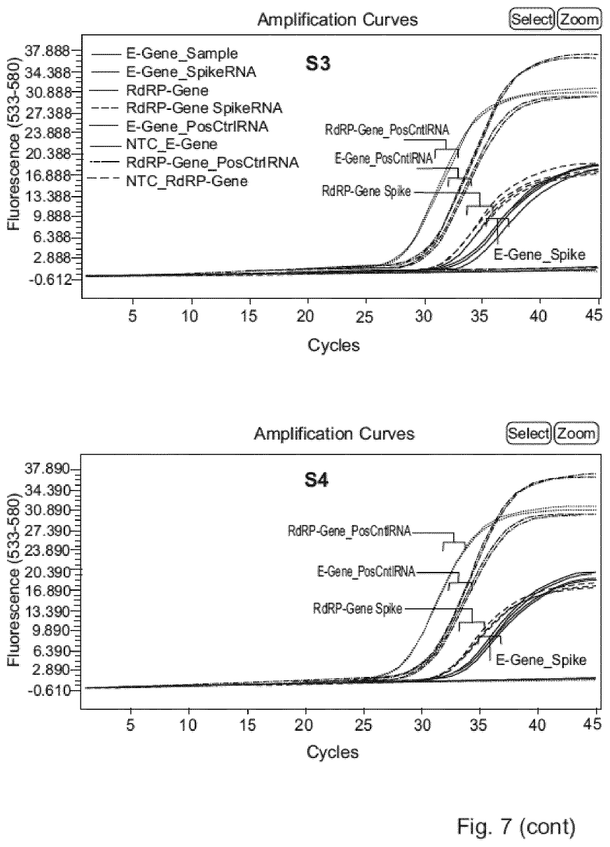

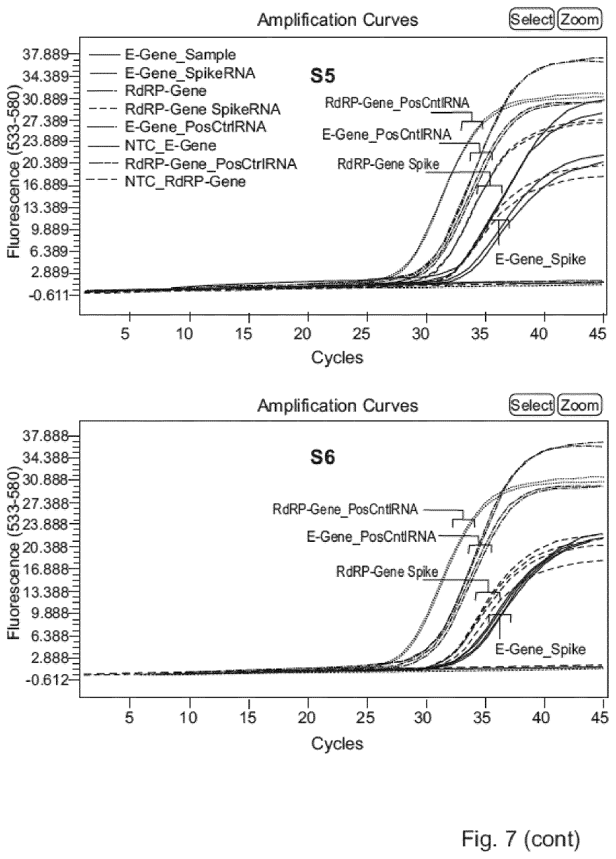

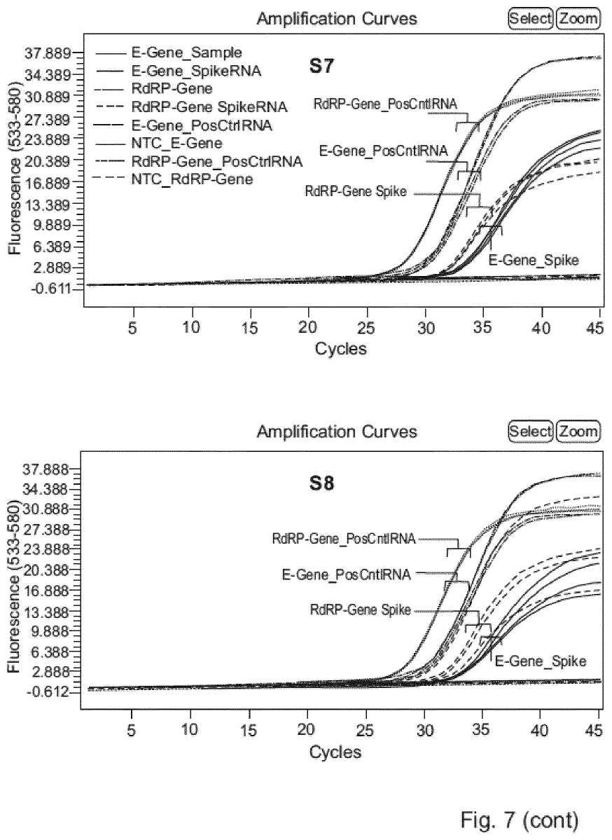

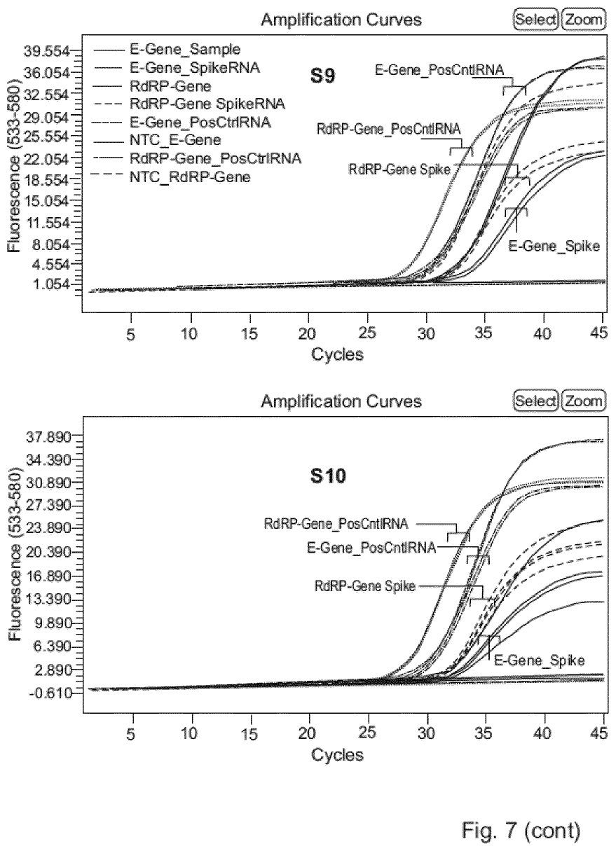

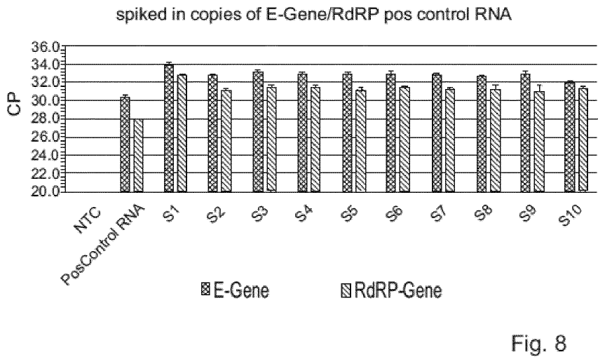

Amplification curves from each sample are provided in FIG. 7 and summarized in FIG. 8. Data are presented below in Table 6.

TABLE 6

| | CP values | | Stddev (CP) | |
|---|---|---|---|---|
| NTC | E-Gene | RdRP-Gene | E-Gene | RdRP-Gene |
| PosCtrl RNA | 30.43 | 27.94 | 0.16 | 0.04 |
| S1 | 33.97 | 32.73 | 0.25 | 0.12 |
| S2 | 32.75 | 31.13 | 0.20 | 0.18 |
| S3 | 32.15 | 31.46 | 0.27 | 0.24 |
| S4 | 32.96 | 31.59 | 0.13 | 0.14 |
| S5 | 32.90 | 31.04 | 0.22 | 0.47 |
| S6 | 32.94 | 31.38 | 0.28 | 0.09 |
| S7 | 32.97 | 31.29 | 0.14 | 0.18 |
| S8 | 32.70 | 31.24 | 0.09 | 0.43 |
| S9 | 32.97 | 31.09 | 0.26 | 0.62 |
| S10 | 32.06 | 31.37 | 0.10 | 0.15 |

These data illustrate that SARS CoV-2 genes can be reliably detected from nasal swab samples prepared without inclusion of an RNA extraction step. Further studies will be conducted to demonstrate that viral RNA present in nasal swabs of CoV-2 infected patients can be measured by the described method.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1: A method to detect an RNA or DNA virus comprising contacting at least one sample with a lysis buffer to produce a lysate, in case of RNA virus detection reverse transcribing RNA within the lysate to obtain cDNA, and amplifying at least one nucleic acid from an RNA or DNA virus in the lysate using a set of primers derived from a nucleic acid sequence of the RNA or DNA virus,
wherein the lysis buffer comprises at least one non-ionic surfactant, glycerol, and at least one salt,
wherein the at least one salt is disodium phosphate, and
wherein the method does not comprise an RNA extraction step.

2. (canceled)

3: The method according to claim 1, wherein the at least one non-ionic surfactant has a hydrophilic polyethylene oxide chain and an aromatic hydrocarbon lipophilic or hydrophobic group.

4: The method according to claim 1, wherein the at least one non-ionic surfactant is Triton X-100.

5: The method according to claim 1, wherein the lysis buffer comprises a non-ionic surfactant in an amount from about 0.05% to about 20%.

6. (canceled)

7: The method according to claim 1, wherein the lysis buffer further comprises one or more of the following: Tris-HCl, dithiothreitol (DTT), RNase-free water, RNase inhibitor, and mixtures thereof.

8: The method according to claim 1, wherein the pH of the lysis buffer is from about 7.5 to about 8.5.

9: The method according to claim 1, wherein the RNA virus is a coronavirus.

10: The method according to claim 9, wherein the coronavirus is SARS CoV-2 or a variant of SARS CoV-2.

11-15. (canceled)

16: The method according to claim 1, wherein the lysate is directly used in the reverse transcription.

17: The method according to claim 1, wherein the sample is a biological sample.

18: The method according to claim 17, wherein the biological sample is collected using a swab.

19: The method according to claim 17, wherein the biological sample is a nasal swab.

20-51. (canceled)

52: A kit comprising a lysis buffer, wherein the lysis buffer comprises at least one non-ionic surfactant, glycerol, and at least one salt, and at least one reagent for amplification of a target nucleic acid.

53. (canceled)

54: The kit according to claim 52, comprising at least one primer and/or at least one probe for amplification of a target nucleic acid.

55: The kit according to claim 52, wherein the kit is a kit for detection of RNA using one-step RT-qPCR.

56. (canceled)

57: The kit according to claim 52, wherein the target nucleic acid is derived from a RNA virus.

58: The kit according to claim 57, wherein the RNA virus is a coronavirus.

59: The kit according to claim 57, wherein the RNA virus is SARS CoV-2.

60-66. (canceled)

67: Use of the lysis buffer comprising 1-5% Triton X-100 and one or more of the following components: 5-20% glycerol, 0.5-4 mM DTT, 10-50 mM Na₂HPO₄, and 10-50 mM Tris-HCl in a method for amplifying one or more nucleic acids comprising contacting at least one sample with the lysis buffer to produce a lysate, and amplifying at least one nucleic acid in the lysate.

68: Use of the lysis buffer comprising 1-5% Triton X-100 and one or more of the following components: 5-20% glycerol, 0.5-4 mM DTT, 10-50 mM Na₂HPO₄, and 10-50 mM Tris-HCl in a method to detect an RNA virus comprising contacting at least one sample with the lysis buffer to produce a lysate, reverse transcribing RNA within the lysate to obtain cDNA, and amplifying at least one nucleic acid from an RNA virus in the lysate using a set of primers derived from a nucleic acid sequence of the RNA virus.

69: The use according to claim 67, wherein the lysis buffer comprises about 3% Triton X-100, about 10% glycerol, about 2 mM DTT, about 25 mM Na₂HPO₄, and about 25 mM Tris-HCl.

70: The use according to claim 68, wherein the lysis buffer comprises about 3% Triton X-100, about 10% glycerol, about 2 mM DTT, about 25 mM Na₂HPO₄, and about 25 mM Tris-HCl.
